SEQUENCE LISTING (1 sequence)

SEQ ID NO: 1 - protein, 87 aa, synthetic construct
STSEDQYYDTNYDGQVDTVVTDTDGNGVYDAAVYDTDGNGVADTVAYDSDENGVVDTVGFDYNEDGVVDEVVTDYNEDGYADSSSSS

(19) United States
(12) Patent Application Publication
(10) Pub. No.: US 2026/0103486 A1
(43) Pub. Date: Apr. 16, 2026

(54) RARE EARTH ELEMENT BINDING PROTEIN

(71) Applicant: Battelle Memorial Institute, Columbus, OH (US)

(72) Inventors: Bradley S. HEATER, Dublin, OH (US); Katarzyna H. KUCHARZYK, Columbus, OH (US); Daniel R. MARZOLF, Columbus, OH (US); Steven A. HIGGINS, Columbus, OH (US); Jonathan W. PICKING, Hilliard, OH (US); Scott BANTA, Fairfield, CT (US); Farid KHOURY, New York, NY (US)

(21) Appl. No.: 19/339,618

(22) Filed: Sep. 25, 2025

Related U.S. Application Data
(60) Provisional application No. 63/698,701, filed on Sep. 25, 2024.

(51) Int. Cl. C07K 7/06; C01F 17/10

(52) U.S. Cl. CPC C07K 7/06; C01F 17/10

(57) ABSTRACT

This invention relates to rare earth element binding protein and methods of recovering a rare earth element (REE) from a sample.

### STATEMENT REGARDING FEDERALLY SPONSORED RESEARCH OR DEVELOPMENT

This invention was made with government support under Contract Number FA8650-22-C-7213 awarded by the Defense Advanced Research Projects Agency (DARPA). The government has certain rights in the invention.

### SEQUENCE LISTING

The instant application contains a Sequence Listing which has been submitted electronically in XML format and is hereby incorporated by reference in its entirety. Said XML copy, created on Sep. 12, 2025 is named BAT299US SL.xml and is 1772 bytes in size.

### FIELD

This invention relates to a rare earth element binding protein and methods of recovering a rare earth element (REE) from a sample.

### BACKGROUND

A challenge in establishing a more diversified REE supply chain is the difficulty of achieving cost-effective and environmentally sustainable REE extraction and separation from ore deposits and REE-containing waste. The current industrial REE production processes generate radioactive wastes, high volumes of acidic effluents, and organic solvents, resulting in a severe environmental burden. To alleviate supply vulnerability and diversify the global REE production chain, new processing technologies, specifically based in biological advancements enabling green REE extraction from alternative REE resources, are desired.

Proteins offer highly specific environments for metal-biological interactions to occur, so there is significant interest in their use for eco-friendly REE separation and purification. One such protein of interest is Lanmodulin (LanM), which reportedly demonstrates significantly higher binding affinity for REEs compared to calcium and other contaminant metals. However, LanM only binds 1-2 REEs when immobilized and exhibits relatively poor selectivity for individual REEs. See, e.g., WO2022/266120 entitled Compositions Comprising Proteins And Methods OF Use Thereof For Rare Earth Element Separation.

### SUMMARY

A rare earth element (REE) binding protein comprising the repeating sequence X₁X₂X₃X₄X₅X₆X₇X₈X₉ wherein X denotes any amino acid, and X₁ is D, X₂ is Y, T, or S, X₃ is D or N, X₄ is G or E, X₅ is D or N, X₆ is G, X₇ is Y, Q or V, X₈ is A, V, or Y, X₉ is D.

A rare earth element (REE) binding protein wherein the REE binding protein comprises a sequence with at least 75% identity to SEQ ID NO. 1.

A method of recovering a rare earth element (REE) from a sample comprising:
a. introducing a REE binding protein to the sample wherein the REE binding protein comprises the repeating sequence X₁X₂X₃X₄X₅X₆X₇X₈X₉ wherein X denotes any amino acid, and X₁ is D, X₂ is Y, T, or S, X₃ is D or N, X₄ is G or E, X₅ is D or N, X₆ is G, X₇ is Y, Q or V, X₈ is A, V, or Y, X₉ is D; and
b. recovering the REE from the sample.

A method of recovering a rare earth element (REE) from a sample comprising:
a. introducing a REE binding protein to the sample wherein the REE binding protein comprises a sequence with at least 75% identity to SEQ ID NO. 1; and
b. recovering the REE from the sample.

A method of recovering a rare earth element (REE) from a sample comprising:
a. providing REE binding protein immobilized on a solid matrix where the REE binding protein comprises the repeating sequence X₁X₂X₃X₄X₅X₆X₇X₈X₉ wherein X denotes any amino acid, and X₁ is D, X₂ is Y, T, or S, X₃ is D or N, X₄ is G or E, X₅ is D or N, X₆ is G, X₇ is Y, Q or V, X₈ is A, V, or Y, X₉ is D;
b. introducing a sample containing one or more REEs onto said REE binding protein immobilized on said solid matrix;
c. loading said one or more REEs onto said REE binding protein immobilized on said solid matrix; and
d. unloading said one or more REEs from said REE binding protein immobilized on said solid matrix.

A method of recovering a rare earth element (REE) from a sample comprising:
a. providing REE binding protein immobilized on a solid matrix where the REE binding protein comprises a sequence with at least 75% identity to SEQ ID NO. 1
b. introducing a sample containing one or more REEs onto said REE binding protein immobilized on said solid matrix;
c. loading said one or more REEs onto said REE binding protein immobilized on said solid matrix; and
d. unloading said one or more REEs from said REE binding protein immobilized on said solid matrix.

### DRAWINGS

FIG. 1 provides an AlaphaFold3 model of the REE binding protein comprising SEQ ID NO. 1 bound to calcium.

FIG. 2 provides a plot of the dissociation constant for the indicated REE binding proteins HEW5 and A0A7 for the indicated rare earth elements La, Ce, Pr, Nd, Eu, Tb, Dy, Yb and Lu.

FIG. 3 shows La binding to immobilized Halo-HEW5 or Halo-A0A7 at 10 and 60 mg/mL loading in spin column format. FT: Flowthrough; W-Wash; E-Elution.

FIG. 4 shows the fraction of REE desorbed versus elution volume (mL) for the indicated rare earth elements.

FIG. 5 shows circular dichroism spectra (CD) spectroscopy at pH 6 of A0A7 (60 μM) in its apo, Ca-bound and Nd-bound states.

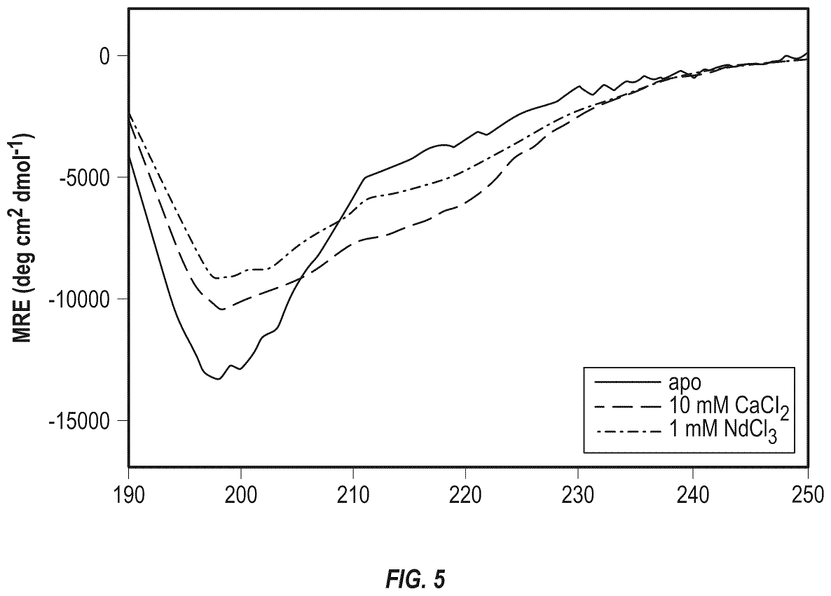

FIG. 6 shows the selectivity of A0A7, HEW5 and RTX for lanthanides over calcium and maximum selectivity within the lanthanide series.

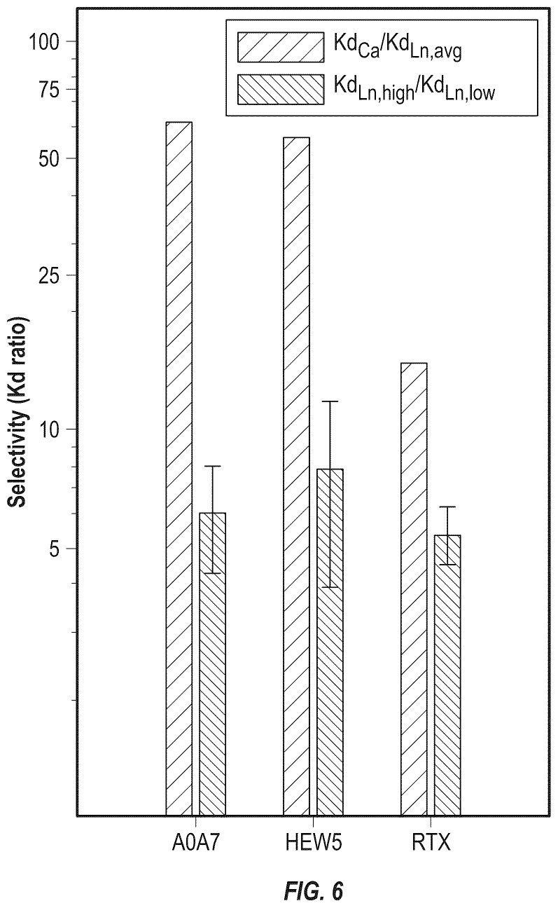

FIG. 7 shows the immobilized apparent dissociation constants measured under isocratic elution conditions (pH 3) for A0A7 and HEW5.

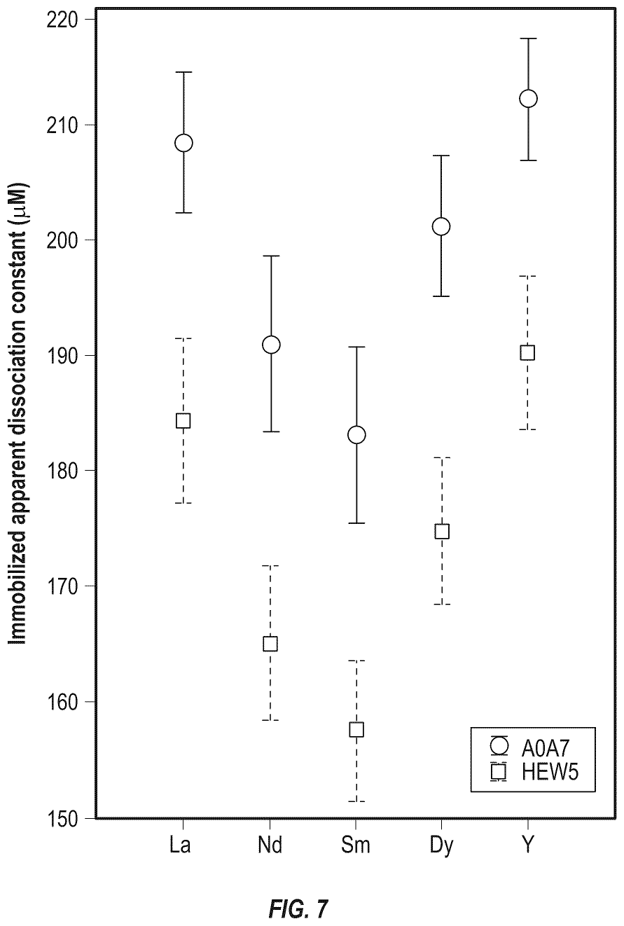

### DETAILED DESCRIPTION OF PREFERRED EMBODIMENTS

REEs comprise a group of metals including lanthanides, yttrium (Y), and scandium (Sc). The lanthanides (or lanthanoids) are elements with atomic numbers 57 through 71 (e.g., lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Flo), erbium (Er), thulium I, ytterbium (Yb), and lutetium (Lu), respectively).

The present invention provides a REE binding protein, which may be utilized for example, in the recovery of REEs from a sample. The protein is contemplated to bind REEs in the range of 10 μM to 50 μM. In addition, the REE binding protein is contemplated to preferably provide a selectivity toward light rare earth elements (i.e., La, Ce, Pr, Nd, Pm, Sm, Eu, Gd) as compared to heavy rare earth elements (i.e., Tb, Dy, Ho, Er, Tm, Yb, Lu). More preferably, the preference amounts to a 2-4 fold selectivity preference toward light REE compared to heavy REE.

The REE binding protein herein may first be described as continuous sequence of at least nine (9) amino acids with the sequence X₁X₂X₃X₄X₅X₆X₇X₈X₉ wherein X denotes any amino acid, and X₁ is D, X₂ is Y, T, or S, X₃ is D or N, X₄ is G or E, X₅ is D or N, X₆ is G, X₇ is Y, Q or V, X₈ is A, V, or Y, X₉ is D. More preferably, the aforementioned sequence is contemplated to repeat 2, 4, 6, 8, 10 or 12 times, and such repeating sequence is preferably separated by at least four (4) amino acids.

The REE binding protein can also preferably be described as having the domain sequence selected from SEQ ID NO.1, also identified herein as A0A7:

STSEDQYYDTNYDGQVDTVVTDTDGNGVYDAAVYDTDGNGVADTV
AYDSDENGVVDTVGFDYNEDGVVDEVVTDYNEDGYADSSSSS

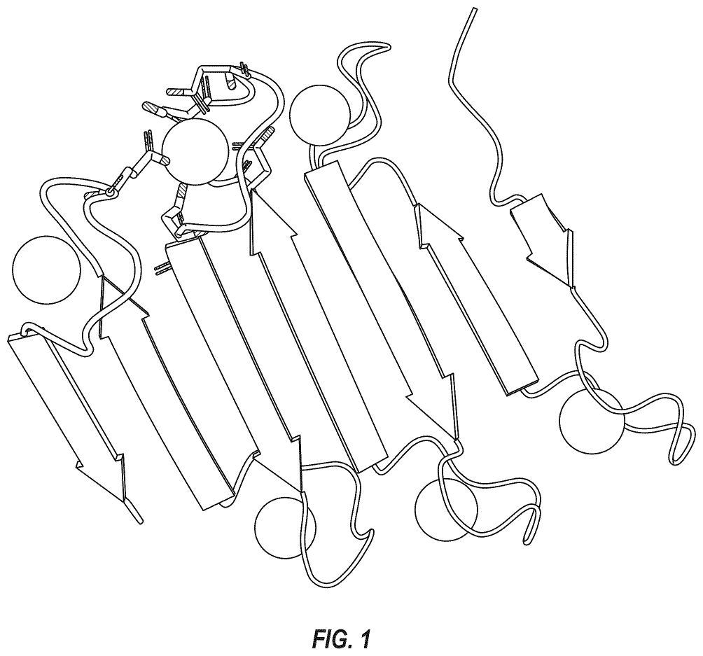

In certain embodiments, the REE binding protein comprises, consists of, or consists essentially of the amino acid sequence set forth in SEQ ID NO. 1, or a sequence with at least 75%, 80%, 85%, 90%, 91%, 92%, 93%, 94%, 95%, 96%, 97%, 98%, or 99% identity to SEQ ID NO. 1. The underlined sequences identified in SEQ ID NO. 1 identify six (6) sequence motifs that are contemplated to provide the REE binding selectivity disclosed herein. FIG. 1 provides an AlphaFold3 model of the REE binding protein herein comprising SEQ ID NO. 1.

The REE binding protein described herein is therefore contemplated for use in a method to recover a REE from a sample. This is contemplated to involve introducing a REE binding protein to the sample wherein the REE binding protein again comprises the repeating sequence X₁X₂X₃X₄X₅X₆X₇X₈X₉ wherein X denotes any amino acid, and X₁ is D, X₂ is Y, T, or S, X₃ is D or or N, X₄ is G or E, X₅ is D or N, X₆ is G, X₇ is Y, Q or V, X₈ is A, V, or Y, X₉ is D. This is followed by recovery of the REE from the sample.

The method to recover a REE from a sample is also contemplated to involve introducing the REE binding protein to a sample, where the REE binding protein comprises a sequence with at least 75% identity to SEQ ID NO. 1, and recovering the REE from the sample.

The method to recover a REE from a sample is also contemplated to involve providing a REE binding protein immobilized on a solid matrix where the REE binding protein again comprises the repeating sequence X₁X₂X₃X₄X₅X₆X₇X₈X₉ wherein X denotes any amino acid, and X₁ is D, X₂ is Y, T, or S, X₃ is D or N, X₄ is G or E, X₅ is D or N, X₆ is G, X₇ is Y, Q or V, X₈ is A, V, or Y, X₉ is D. This is followed by introducing a sample containing one or more REEs onto the REE binding protein immobilized on the solid matrix; loading the one or more REEs onto the REE binding protein immobilized on the solid matrix; unloading the one or more REEs from the REE binding protein immobilized on the solid matrix. The REE binding protein may be immobilized on the solid matrix via a non-covalent or covalent bond linkage.

The method to recover a rare earth element (REE) from a sample is further contemplated to involve providing REE binding protein immobilized on a solid matrix where the REE binding protein comprises a sequence with at least 75% identity to SEQ ID NO. 1. This is followed by introducing a sample containing one or more REEs onto said REE binding protein immobilized on the solid matrix; loading the one or more REEs onto the REE binding protein immobilized on the solid matrix; and unloading the one or more REEs from said REE binding protein immobilized on the solid matrix. The REE binding protein may be immobilized on the solid matrix via a non-covalent or covalent bond linkage.

One of the advantages of using the REE binding protein herein for REE separation is that a relatively weak interaction is present as between the REE binding protein and the REE targeted for separation and recovery. This then allows for relatively facile release of the REE from the binding protein under what can be characterized as relatively mild conditions. Such mild conditions include conditions such as exposure to a pH ≥3.0, whereas a relatively strong interaction would require a lower pH (<3.0) such as 1.5 to remove the REE from the protein. Removal of the REE from the binding protein herein is also contemplated to make use of a relatively weak chelator (such as acetate, sulphate, formate) which relatively weak chelator can then itself be more easily removed from the REE. By contrast, a relatively strong chelator (such as citrate, EDTA or EGTA) which is required to remove REEs from a protein with a relatively strong interaction) is more difficult to separate from the REE without precipitating the REE out of solution.

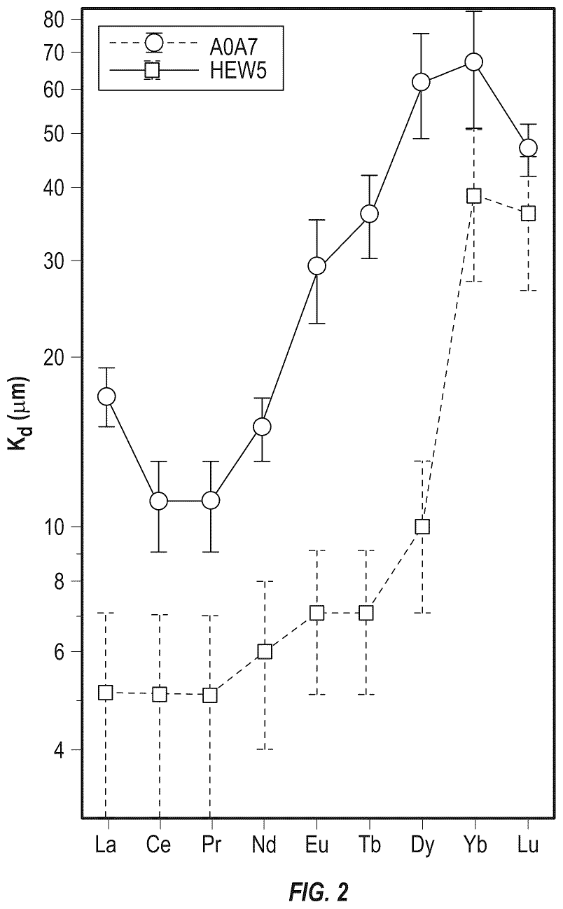

Isothermal titration calorimetry (ITC), which measures heat release after a binding event and is used to characterize protein-ligand interactions, was employed to determine dissociation constants of different REE-protein interaction. Reference is made to FIG. 2, which shows the results of such ITC evaluation on two proteins, namely a protein identified as HEW5 and the protein herein now labelled as A0A7. It is worth noting that the protein HEW5 is also a continuous sequence of at least nine (9) amino acids with the sequence X₁X₂X₃X₄X₅X₆X₇X₈X₉ wherein X denotes any amino acid, and X₁ is D or E, X₂ is A, T, or S, X₃ is D, E or N, X₄ is G, A, or F, X₅ is D, X₆ is G, S, or D, X₇ is Y, L, V, F, I, E or W, X₈ is A, V, I, L, F or T, X₉ is D, E, or N. As can be seen, although the binding affinity of HEW5 to the REEs was relatively stronger than the protein herein identified as A0A7, the trend is similar.

The A0A7 protein herein may be further characterized as follows. As noted, it has 87 residues along with a molecular weight of 9.3 kDa, a pI value (isoelectric point) of 2.69. The number of theoretical binding sites was determined to be six (6) and the capacity (#of theoretical binding sites/100 AA) was determined to be 6.

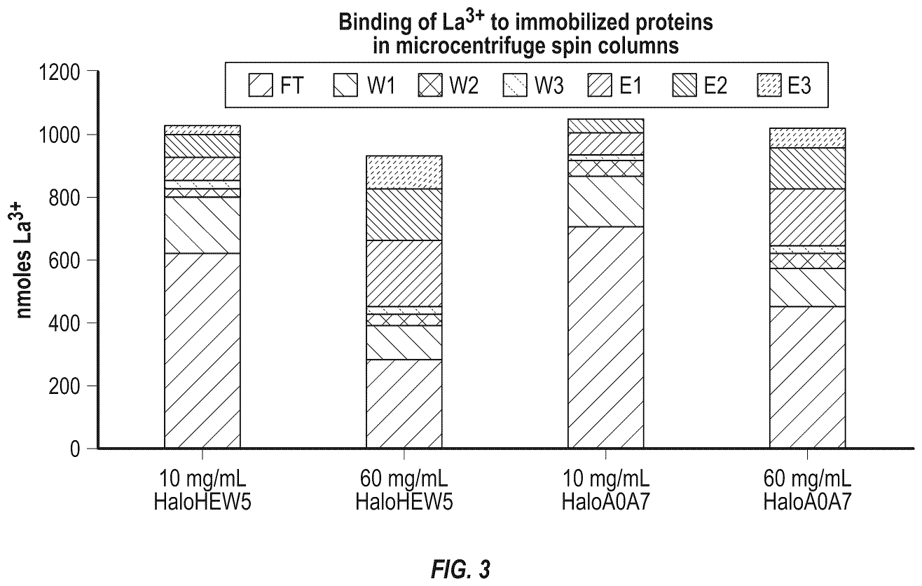

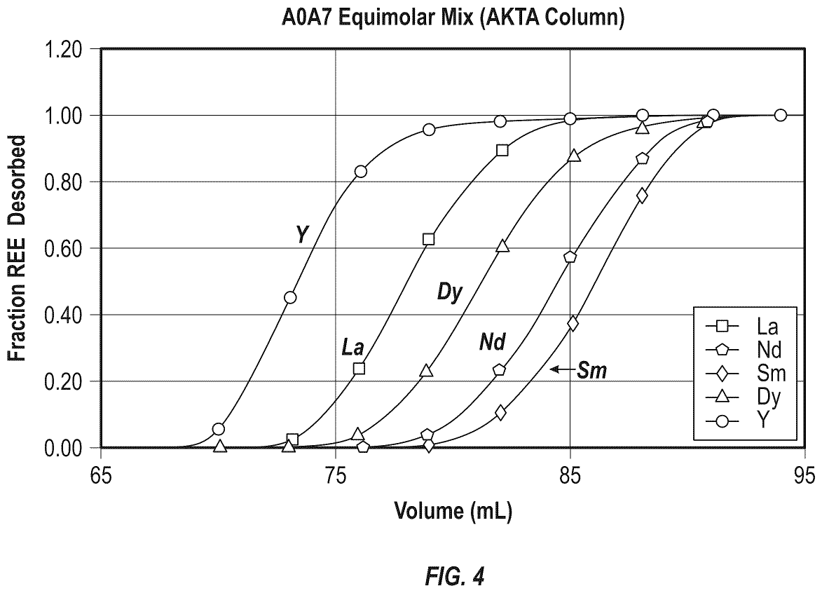

Immobilization of the protein herein onto a solid matrix, such as beads, was achieved utilizing Halotag-Halolink chemistry. An arsenazo assay was performed to assess La binding to immobilized Halo-HEW5 or Halo-A0A7 at 10 and 60 mg/mL loading in spin column format. See FIG. 3. This data demonstrates that the protein herein, identified as A0A7 binds La³⁺. Importantly, immobilized A0A7 could also separate a mixture of 5×REEs by applying REEs to the column in 20 mM MES buffer pH 5.5 and eluting at pH 3.0. See FIG. 4.

The production of the REE binding protein herein can preferably be achieved via nucleic acid encoding. Namely, nucleic acid encoding of the REE binding protein described herein as: (1) nine (9) amino acids with the sequence X₁X₂X₃X₄X₅X₆X₇X₈X₉ wherein X denotes any amino acid, and X₁ is D or E, X₂ is A, T, or S, X₃ is D, E or N, X₄ is G, A, or F, X₅ is D, X₆ is G, S, or D, X₇ is Y, L, V, F, I, E or W, X₈ is A, V, I, L, F or T, X₉ is D, E, or N; or (2) a protein with at least 75% identity to SEQ ID NO. 1.

Because of the knowledge of the codons corresponding to the various amino acids, availability of an amino acid sequence of a polypeptide of interest provides a description of all the polynucleotides capable of encoding the polypeptide of interest. The degeneracy of the genetic code, where the same amino acids are encoded by alternative or synonymous codons allows an extremely large number of nucleic acids to be made, all of which encode the REE binding proteins disclosed herein. Thus, having identified a particular amino acid sequence, those of ordinary skill in the art could make any number of different nucleic acids by modifying the sequence of one or more codons in a way which does not change the amino acid sequence of the REE binding protein herein of interest. In this regard, the present disclosure specifically contemplates each and every possible variation of polynucleotides that could be made by selecting combinations based upon the possible codon choices, and all such variations are to be considered specifically disclosed for any REE binding protein disclosed herein.

The nucleotide sequences of the nucleic acids of the present disclosure may be codon-optimized. “Codon-optimized” refers to changes in the codons of the polynucleotide encoding a polypeptide to those preferentially used in a particular organism such that the encoded protein is efficiently expressed in the organism of interest. Although the genetic code is degenerate in that most amino acids are represented by several codons, called “synonyms” or “synonymous” codons, it is known that codon usage by particular organisms is nonrandom and biased towards particular codon triplets. This codon usage bias may be higher in reference to a given gene, genes of common function or ancestral origin, highly expressed proteins versus low copy number proteins, and the aggregate protein coding regions of an organism's genome. In some embodiments, a nucleic acid of the present disclosure encoding a polypeptide may be codon-optimized for optimal production from the host organism selected for expression, e.g., a prokaryote.

Aspects of the present disclosure further include expression constructs comprising a nucleic acid of the present disclosure operably linked to a promoter. As used herein, an “expression construct” is a circular or linear polynucleotide (a polymer composed of naturally-occurring and/or non-naturally-occurring nucleotides) comprising a region that encodes the REE binding protein operably linked to a suitable promoter, e.g., a constitutive or inducible promoter. In some embodiments, expression of the REE binding protein herein is preferably under the control of one or more exogenous (including heterologous) regulatory elements, e.g., promoter, enhancer, etc., present in the expression construct, and operably linked to the region encoding the REE binding protein. In some embodiments, expression of the REE binding protein may be preferably controlled by one or more endogenous regulatory elements, e.g., promoter, enhancer, etc., at or near a genomic locus into which the expression construct is inserted.

The expression construct (e.g., vector) can be suitable for replication and integration in prokaryotes, eukaryotes, or both. The expression constructs may contain functionally appropriately oriented transcription and translation terminators, initiation sequences, and promoters useful for regulation of the expression of the nucleic acid encoding the REE binding protein herein. The expression construct optionally contains generic expression cassettes containing at least one independent terminator sequence, sequences permitting replication of the cassette in both eukaryotes and prokaryotes, e.g., as found in shuttle vectors, and selection markers for both prokaryotic and eukaryotic systems.

To obtain relatively high levels of expression of a cloned nucleic acid it is common to construct expression constructs which typically contain a strong promoter to direct transcription, a ribosome binding site for translational initiation, and a transcription/translation terminator, each in functional orientation to each other and to the protein-encoding sequence. The inclusion of selection markers in DNA vectors is also useful. Examples of such markers include genes specifying resistance to ampicillin, tetracycline, or chloramphenicol. Transducing cells with nucleic acids can involve, for example, incubating lipidic microparticles containing nucleic acids with cells or incubating viral vectors containing nucleic acids with cells within the host range of the vector.

In certain embodiments, upon delivery of the expression construct into a population of cells of interest, the expression construct is episomal (e.g., extra-chromosomal), whereby “episome” or “episomal” is meant a polynucleotide that replicates independently of the cell's chromosomal DNA. A non-limiting example of an episome that may be employed is a plasmid.

According to some embodiments, upon delivery of the expression construct into a population of cells of interest, the expression construct integrates into the genome of the cell. In certain embodiments, the expression construct is adapted for site-specific integration into the genome. For example, an expression construct may be adapted for site-specific integration into the genome, where the site-specific integration inactivates a target gene within the genome of the cell. By way of example, the site-specific integration may knock-out the target gene by knock-in of the expression construct. Any suitable approach for site-specific gene editing and functional integration may be employed. Functional integration of an expression construct may be achieved through various means, including through the use of integrating vectors, including viral and non-viral vectors. In some instances, a retroviral vector, e.g., a lentiviral vector, may be employed. In some instances, a non-retroviral integrating vector may be employed. An integrating vector may be contacted with the cells in a suitable transduction medium, at a suitable concentration (or multiplicity of infection), and for a suitable time for the vector to infect the target cells, facilitating functional integration of the expression construct. Non-limiting examples of useful viral vectors include retroviral vectors, lentiviral vectors, adenoviral (Ad) vectors, adeno-associated virus (AAV) vectors, hybrid Ad-AAV vector systems, and the like.

Strategies for site-specific integration that find use include those that employ homologous recombination, nonhomologous end-joining (NHEJ), and/or the like. Such strategies may employ a non-naturally occurring or engineered nuclease, including, but not limited to, zinc-ringer nucleases (ZNFs), meganucleases, transcription activator-like effector nucleases (TALENs)), or a CRISPR-Cas system. Eukaryotic cells utilize two distinct DNA repair mechanisms in response to DNA double strand breaks (DSBs): Homologous recombination (HR) and nonhomologous end-joining (NHEJ). Mechanistically, HR is an error-free DNA repair mechanism because it requires a homologous template to repair the damaged DNA strand. Because of its homology-based mechanism, HR has been used as a tool to site-specifically engineer the genome. Gene targeting by HR requires the use of two homology arms that flank the transgene/target site of interest. HR efficiency can be increased by the introduction of DSBs at the target site using specific rare-cutting endonucleases. The discovery of this phenomenon prompted the development of methods to create site-specific DSBs in the genome of different species. Various chimeric enzymes have been designed for this purpose over the last decade, namely ZFNs, meganucleases, and TALENs. ZFNs are modular chimeric proteins that contain a ZF-based DNA binding domain (DBD) and a FokI nuclease domain. DBD is usually composed of three ZF domains, each with 3-base pair specificity; the FokI nuclease domain provides a DNA nicking activity, which is targeted by two flanking ZFNs. Owing to the modular nature of the DBD, any site in a genome could be targeted. TALENS are similar to ZFNs except that the DBD is derived from transcription activator-like effectors (TALEs). The TALE DBD is modular, and it is composed of 34-residue repeats, and its DNA specificity is determined by the number and order of repeats. Each repeat binds a single nucleotide in the target sequence through only two residues.

Accordingly, aspects of the present disclosure further include a cell or population of cells comprising any of the nucleic acids or expression constructs of the present disclosure. In certain embodiments, the population of cells is a population of prokaryotic cells.

A variety of suitable approaches and conditions for the delivery of the expression construct to cells are known. According to some embodiments, the expression construct is delivered to the population of cells by microinjection, transfection, lipofection, heat-shock, electroporation, transduction, gene gun, DEAE-dextran-mediated transfer, and/or the like.

1. A rare earth element (REE) binding protein comprising a repeating sequence X₁X₂X₃X₄X₅X₆X₇X₈X₉, wherein X denotes any amino acid, and X₁ is D, X₂ is Y, T, or S, X₃ is D or N, X₄ is G or E, X₅ is D or N, X₆ is G, X₇ is Y, Q or V, X₈ is A, V, or Y, X₉ is D.

2. A rare earth element (REE) binding protein wherein the REE binding protein comprises a sequence with at least 75% identity to SEQ ID. NO. 1.

3. The REE binding protein of claim 1 or 2 wherein the REE binding protein is immobilized on a solid matrix.

4. The REE binding protein of claim 1 wherein said sequence repeats 2, 4, 6, 8, 10 or 12 times.

5. The REE binding protecting of claim 1 wherein the sequence is separated by at least four (4) amino acids.

6. The REE binding protein of claim 1 or 2 wherein the REE binding protein is immobilized on a solid matrix via a non-covalent linkage.

7. The REE binding protein of claim 1 or 2 wherein the REE binding protein is immobilized on a solid matrix via a covalent linkage.

8. A method of recovering a rare earth element (REE) from a sample comprising:
a. introducing a REE binding protein to the sample wherein the REE binding protein comprises a repeating sequence X₁X₂X₃X₄X₅X₆X₇X₈X₉ wherein X denotes any amino acid, and X₁ is D, X₂ is Y, T, or S, X₃ is D or N, X₄ is G or E, X₅ is D or N, X₆ is G, X₇ is Y, Q or V, X₈ is A, V, or Y, X₉ is D;
b. recovering the REE from the sample.

9. The method of claim 7 further comprising purifying a REE from the sample.

10. The method of claim 7 wherein the REE binding protein indicates a difference in selectivity toward light REEs versus heavy REEs.

11. The method of claim 7 wherein said repeating sequence repeats 2, 4, 6, 8, 10 or 12 times.

12. The method of claim 7 wherein the repeating sequence is separated by at least four (4) amino acids.

13. The method of claim 10 wherein the light REE comprises La, Ce, Pr, Nd, Pm, Sm, Eu, or Gd.

14. The method of claim 10 wherein the REE comprises a heavy rare earth element comprising Tb, Dy, Ho, Er, Tm, Yb, or Lu.

15. A method of recovering a rare earth element (REE) from a sample comprising:
a. introducing a REE binding protein to the sample wherein the REE binding protein comprises a sequence with at least 75% identity to SEQ ID NO. 1; and
b. recovering the REE from the sample.

16. The method of claim 16 wherein the REE binding protein indicates a difference in selectivity toward light REEs versus heavy REEs.

17. The method of claim 16 wherein the light REE comprises La, Ce, Pr, Nd, Pm, Sm, Eu, or Gd.

18. The method of claim 16 wherein the heavy REE comprises Tb, Dy, Ho, Er, Tm, Yb, or Lu.

19. A method of recovering a rare earth element (REE) from a sample comprising:
a. providing REE binding protein immobilized on a solid matrix where the REE binding protein comprises a repeating sequence X₁X₂X₃X₄X₅X₆X₇X₈X₉ X denotes any amino acid, and X₁ is D, X₂ is Y, T, or S, X₃ is D or N, X₄ is G or E, X₅ is D or N, X₆ is G, X₇ is Y, Q or V, X₈ is A, V, or Y, X₉ is D;
b. introducing a sample containing one or more REEs onto said REE binding protein immobilized on said solid matrix;
c. loading said one or more REEs onto said REE binding protein immobilized on said solid matrix;
d. unloading said one or more REEs from said REE binding protein immobilized on said solid matrix.

20. The method of claim 20 wherein said unloading of said one or more REEs from said REE binding protein comprises adjusting pH.

21. A method of recovering a rare earth element (REE) from a sample comprising:
a. providing REE binding protein immobilized on a solid matrix where the REE binding protein comprises a sequence with at least 75% identity to SEQ ID NO. 1
b. introducing a sample containing one or more REEs onto said REE binding protein immobilized on said solid matrix;
c. loading said one or more REEs onto said REE binding protein immobilized on said solid matrix; and
d. unloading said one or more REEs from said REE binding protein immobilized on said solid matrix.

22. The method of claim 22 wherein said unloading of said one or more REEs from said REE binding protein comprises adjusting pH.
